(19) United States
(12) Patent Application Publication
(10) Pub. No.: US 2021/0139556 A1
(43) Pub. Date: May 13, 2021

(54) CHIMERIC NOTCH RECEPTORS

(71) Applicant: Stichting Sanquin Bloedvoorziening, Amsterdam (NL)

(72) Inventor: Derk AMSEN, Amsterdam (NL)

(21) Appl. No.: 17/045,829

(22) PCT Filed: Apr. 10, 2019
(86) PCT No.: PCT/NL2019/050212

§ 371 Date: Oct. 7, 2020

(30) Foreign Application Priority Data
Apr. 10, 2018 (EP) 18166498.8

(51) Int. Cl. C07K 14/705; C12N 5/0783; A61K 35/17

(52) U.S. Cl. CPC C07K 14/705; C12N 5/0638; C12N 2510/00; C07K 2319/03; C07K 2319/70; A61K 35/17

(57) ABSTRACT

The invention relates to chimeric receptors comprising an intracellular domain, and transmembrane domain of a Notch receptor and a heterologous extracellular ligand-binding domain and to uses thereof, specifically in improving T cell function and/or T cell survival, more particularly in cancer therapy.

### FIELD OF THE INVENTION

The invention relates to the field of therapy, specifically cancer therapy, more specifically adoptive T cell immunotherapy.

### BACKGROUND

Remarkable successes have been obtained in tumor therapy by adoptive transfer of in vitro expanded Tumor Infiltrating Lymphocytes (TIL) or T cells expressing chimeric antigen receptors (CAR). CARs contain an ectodomain (a portion of an antibody) specific for antigens found on tumors, coupled to the signaling domains of CD′g and a costimulatory receptor, such as CD28 or 4-1BB (FIG. 1). Expression of CARs in T cells leads to their activation by tumor antigens. Up to 90% complete remissions have been obtained with CAR T cells in certain hematological malignancies. Much less success has been obtained in the treatment of solid tumors. Hence, still many patients are not cured by such treatments. Major hurdles are the suboptimal persistence of transferred T cells and blockade of T cell function by multiple inhibitory receptors (a phenomenon known as exhaustion), which must all be targeted for maximal therapeutic effect. Ideally, anti-tumor T cells would be broadly impervious to suppressive mechanisms and live long enough to achieve complete tumor eradication.

Notch is a cell surface receptor that responds to membrane bound ligands. It signals through a strikingly direct pathway, in which the intracellular domain is cleaved off from the plasma membrane by a γ-secretase and migrates to the nucleus to act as a transcription factor (FIG. 2). Notch is a major regulator of both CD4 and CD8 T cell effector differentiation. It also promotes long term survival of CD4 memory T cells as well of Tissue Resident Memory CD8 T cells, which are emerging as the most effective T cell type against solid tumors. Furthermore, Notch is a major regulator of the CD8 effector T cell gene expression program. Among its direct target genes are those encoding IFNγ, Granzyme B and Perforin, as well as the transcription factors T-bet and Eomesodermin. Mice with T cell specific deficiencies in the Notch pathway are unable to reject model tumors. Vice versa, deliberate activation of Notch promoted tumor rejection in mice. Tumor associated myeloid-derived suppressor cells (MDSC) downregulate Notch expression in T cells, presumably helping tumors escape effective T cell-mediated rejection. Expression of an active Notch allele rendered CD8 T cells insensitive to MDSC mediated suppression.

Recent studies (Morsut et al. 2016 and Roybal et al. 2016) created chimeric receptors containing the transmembrane region and a small part of the extracellular region of Notch. These were coupled to ligand-binding domains from unrelated surface receptors, while the intracellular part of Notch was replaced by an unrelated transactivator (Gal4). Ligand binding by these receptors resulted in 7secretase mediated release of Gal4, which then activated transcription of artificial response genes. Hence, in these receptors both the intracellular effector domain of Notch and the extracellular ligand-binding domain of Notch, and consequently Notch signaling, are no longer present.

There remains a need in the art for new compositions and methods for immunotherapy of tumors, either or not to be used in combination with existing immunotherapy.

### SUMMARY OF THE INVENTION

It is an object of the invention to provide methods for improving T cell function in general, and specifically in tumor immunotherapy.

The invention therefore provides a chimeric receptor comprising an intracellular domain and transmembrane domain of a Notch receptor and a heterologous extracellular ligand-binding domain. The chimeric receptor further preferably comprises a heterodimerization domain and a Lin-12-Notch (LNR) repeats domain of the Notch receptor.

The chimeric receptor according to the invention is capable of Notch signaling, preferably Notch1, Notch2, Notch3 and/or Notch4 signaling, more preferably Notch1 and/or Notch2 signaling, when the heterologous extracellular ligand-binding domain is bound a ligand.

In a further aspect, the invention provides a nucleic acid molecule comprising a sequence encoding a chimeric receptor according to the invention.

In a further aspect, the invention provides a vector comprising a nucleic acid molecule according to the invention.

In a further aspect, the invention provides an isolated cell comprising the nucleic acid molecule according to the invention. In a further aspect, the invention provide a population of such cells.

In a further aspect, the invention provides an isolated cell expressing a chimeric receptor according to the invention. In a further aspect, the invention provide a population of such cells.

In a further aspect, the invention provides a genetically modified T lymphocyte, which is transduced by the nucleic acid molecule or vector of the invention.

In a further aspect, the invention provides a pharmaceutical composition comprising a nucleic acid molecule, vector or cell according the invention and a pharmaceutically acceptable carrier, diluent or excipient.

In a further aspect, the invention provides a method for improving T cell function and/or T cell survival in a subject in need thereof, the method comprising administering to the subject a therapeutically effective amount of a chimeric receptor, a nucleic acid molecule, a vector or a cell according to the invention.

In a further aspect, the invention provides a chimeric receptor, a nucleic acid molecule, a vector or a cell according to the invention for use in a method for improving T cell function and/or T cell survival in a subject.

In a further aspect, the invention provides a method of immunotherapy in a subject in need thereof, the method comprising administering to the subject a therapeutically effective amount of a chimeric receptor, a nucleic acid molecule, a vector or a cell according to the invention.

In a further aspect, the invention provides a chimeric receptor, a nucleic acid molecule, a vector or a cell according to the invention for use in therapy, preferably immunotherapy.

In a further aspect, the invention provides a method for enhancing efficacy of an antibody-based immunotherapy in a subject suffering from cancer and being treated with said antibody, the method comprising administering to the subject a therapeutically effective amount of T cells expressing the chimeric receptor according to the invention.

In a further aspect, the invention provides T cells expressing a chimeric receptor according to the invention for use in a method for enhancing efficacy of an antibody-based immunotherapy in a subject suffering from cancer and being treated with said antibody.

In a further aspect, the invention provides a method of treating cancer in a subject in need thereof, the method comprising administering to the subject an effective amount of T cells comprising a nucleic acid sequence encoding the chimeric receptor according to the invention.

In a further aspect, the invention provides T cells comprising a nucleic acid sequence encoding the chimeric receptor according to the invention for use in a method of treating cancer in a subject.

In a further aspect, the invention provides a method of producing a population of cells according to the invention, comprising

providing cells, preferably human T-cells,

providing said cells with a nucleic acid molecule or vector according to the invention, and

allowing expression of the chimeric antigen receptor according to the invention.

### DETAILED DESCRIPTION

The present invention is concerned with a chimeric receptor with functioning Notch signaling following ligand binding which receptor is created from a combination of the intracellular effector and transmembrane domains of Notch and a heterologous extracellular ligand binding domain. The present inventors found that Notch signaling suppresses expression of T cell specific inhibitory receptors such as PD1 (programmed death protein 1) and LAGS (lymphocyte activation gene 3) on T cells. Tumors often escape immune destruction by reducing the anti-tumor T cell response through upregulation of such inhibitory molecules. Therefore, therapeutic activation of Notch is an attractive target to enhance T cell responses against tumors in human patients. So far, therapeutic use of Notch has been precluded by two problems. First, Notch functions in many cell types and its systemic activation is likely to elicit many side effects. Second, excessive Notch signaling can be oncogenic. Now that the present inventors found that Notch signaling is maintained when combining the intracellular effector domain of Notch with a heterologous extracellular binding domain, these drawbacks are avoided because activation of Notch signaling can be regulated, both in time and location in the body. This is because the chimeric receptor of the invention responds to a heterologous ligand of choice. In the examples the preparation of a chimeric Notch receptor consisting of an ScFv antibody domain directed against human CD19 fused to the 5′end of the human NOTCH1 protein is described.

Hence, the invention provides a chimeric receptor comprising an intracellular domain, and transmembrane domain of a Notch receptor and a heterologous extracellular ligand-binding domain. The chimeric receptor further preferably comprises a heterodimerization domain and a Lin-12-Notch (LNR) repeats domain of the Notch receptor.

The Notch receptors Notch1, Notch2, Notch3 and Notch4 and their sequences are well known in the art, as well as the different domains in these receptors and their sequence, including the Notch intracellular domain, transmembrane domain, heterodimerization domain, Lin-12-Notch (LNR) repeats domain and negative regulatory region (NRR). Hence, a skilled person is well capable of selecting the appropriate domain when making or using a chimeric receptor according to the invention.

An “intracellular domain of a Notch receptor” as used herein refers to an intracellular domain that is capable of initiating Notch1, Notch2, Notch3 or Notch4 signaling, preferably Notch1 or Notch2 signaling. The chimeric receptor according to the present invention is thus capable of Notch signaling, preferably Notch1, Notch2, Notch3 and/or Notch4 signaling, more preferably Notch1 and/or Notch2 signaling. Notch signaling, preferably Notch1, Notch2, Notch3 and/or Notch4 signaling, more preferably Notch1 and/or Notch2 signaling, is induced when the heterologous extracellular ligand-binding domain is bound a ligand. Hence, “capable of Notch signaling” means that Notch signaling is induced when the heterologous extracellular ligand-binding domain of the chimeric repeptor is bound a ligand. The Notch intracellular domain is well known to a person skilled in the art. Preferably it comprises the Notch intracellular domain (NICD), this is the domain that is cleaved of by v-secretase after ligand binding to the Notch extracellular domain of an intact Notch receptor, preferably the NICD of Notch1 or Notch1, more preferably of human Notch1, or a Notch signaling pathway initiating part of the NICD. Said part is capable of initiating Notch signaling. The chimeric receptor furthermore in a preferred embodiment comprises the entire intracellular domain of Notch1, including the C-terminal transactivation domain, the RAM domain and the ankyrin repeats.

The NICD can be used including or lacking the C-terminal PEST region. Truncation of this region results in a more stable NICD protein, which elicits stronger and more sustained signals. Hence, in a particularly preferred embodiment, the intracellular domain of a Notch receptor comprises a sequence of amino acids 1744 to 2424 of the sequence shown in FIG. 8, or the corresponding sequence of a Notch receptor other than Notch 1, or a sequence that is at least 90% identical to said sequence. Said sequence is preferably capable of initiating Notch signaling. Said sequence is preferably at least 95% identical to amino acids 1744 to 2424 of said sequence shown in FIG. 8, more preferably at least 97%, more preferably at least 98%, more preferably at least 99%. In a particularly preferred embodiment, the intracellular domain of a Notch receptor comprises amino acids 1744 to 2424, of the sequence shown in FIG. 8, more preferably it consists of amino acids 1744 to 2424 of the sequence shown in FIG. 8. It is preferred that the intracellular domain comprises the indicated sequence of Notch1, and thus amino acids 1744 to 2424, of the sequence shown in FIG. 8.

In another preferred embodiment, the entire NICD is used, and the intracellular domain of a Notch receptor comprises a sequence of amino acids 1744 to 2555 of the sequence shown in FIG. 8, or the corresponding sequence of a Notch receptor other than Notch 1, or a sequence that is at least 90% identical to said sequence. Said sequence is preferably capable of initiating Notch signaling. Said sequence is preferably at least 95% identical to amino acids 1744 to 2555 of said sequence shown in FIG. 8, more preferably at least 97%, more preferably at least 98%, more preferably at least 99%. In a particularly preferred embodiment, the intracellular domain of a Notch receptor comprises amino acids 1744 to 2555, of the sequence shown in FIG. 8, more preferably it consists of amino acids 1744 to 2555 of the sequence shown in FIG. 8. It is preferred that the intracellular domain comprises the indicated sequence of Notch1, and thus amino acids 1744 to 2555 of the sequence shown in FIG. 8.

A “transmembrane domain” (TMD) of a Notch receptor” as used herein refers to a transmembrane domain of Notch1, Notch2, Notch3 or Notch4, preferably of Notch1 or Notch2. The Notch transmembrane domain is well known to a person skilled in the art. In a particularly preferred embodiment, the transmembrane domain of a Notch receptor comprises a sequence of amino acids 1736 to 1743 of the sequence shown in FIG. 8, or the corresponding sequence of a Notch receptor other than Notch 1, or a sequence that is at least 90% identical to said sequence. Said sequence is preferably capable of initiating cleavage of the NICD by a γ-secretase. Said sequence is further preferably at least 95% identical to amino acids 1736 to 1743 of said sequence shown in FIG. 8, more preferably at least 97%, more preferably at least 98%, more preferably at least 99%. In a particularly preferred embodiment, the transmembrane domain of a Notch receptor comprises amino acids 1736 to 1743 of the sequence shown in FIG. 8, more preferably it consists of amino acids 1736 to 1743 of the sequence shown in FIG. 8. It is preferred that the TMD comprises the indicated sequence of Notch1, and thus amino acids 1736 to 1743 of the sequence shown in FIG. 8.

The heterodimerization domain and Lin-12-Notch (LNR) repeats domain of a Notch receptor together form the negative regulatory region (NRR) of the receptor. The Notch LNR domain, heterodimerization domain and NRR are well known to a person skilled in the art. The heterodimerization domain and the LNR repeats are located between the heterologous extracellular ligand-binding domain and the transmembrane domain in a chimeric receptor of the invention. The order or domains is preferably the following: heterologous extracellular ligand-binding domain—LNR domain—heterodimerization domain—transmembrane domain. Canonical Notch signaling is initiated when a ligand binds to the Notch receptor. This leads to ADAM metalloprotease mediated cleavage of the extracellular fragment of the heterodimer. The membrane tethered fragment is then cleaved by a γ-secretase to release the intracellular fragment of Notch (NICD). The heterodimerization domain and the LNR domain are located in the NRR of the Notch receptor, which is located between the ligand binding domain and the transmembrane domain. The LNRs participate in maintaining the receptor in resting conformation, i.e. prevent or inhibit cleavage by ADAM metalloprotease, in the absence of ligand binding. In a preferred embodiment, the chimeric receptor comprises the entire negative regulatory region (NRR) of the Notch receptor. Preferably this NRR comprises amino acids 1447 to 1735 of the sequence shown in FIG. 8, or the corresponding sequence of a Notch receptor other than Notch 1, or a sequence that is at least 90% identical to said sequence. Said sequence is further preferably at least 95% identical to amino acids 1447 to 1735 of said sequence shown in FIG. 8, more preferably at least 97%, more preferably at least 98%, more preferably at least 99%. In a further preferred embodiment this NRR comprises amino acids 1396 to 1735 of the sequence shown in FIG. 8 or the corresponding sequence of a Notch receptor other than Notch 1, or a sequence that is at least 90% identical to said sequence. Said sequence is further preferably at least 95% identical to amino acids 1447 to 1735 of said sequence shown in FIG. 8, more preferably at least 97%, more preferably at least 98%, more preferably at least 99%. In this sequence, the extracellular portion of the Notch sequence is extended up till proline 1396 (see FIG. 8), as this yields a receptor that is more reliably silent in the absence of ligand binding than shorter constructs. The chimeric receptor of the invention further optionally comprises a signal peptide that directs the receptor to the cell membrane. It is preferred that the NRR comprises the indicated sequence of Notch1, and thus amino acids 1447 to 1735 or 1396 to 1735 of the sequence shown in FIG. 8.

In a particularly preferred embodiment, a chimeric receptor of the invention comprises an intracellular domain, a transmembrane domain, a heterodimerization domain and a Lin-12-Notch (LNR) repeats domain of a Notch receptor and a heterologous extracellular ligand-binding domain, preferably in the indicated order. Hence, a preferred chimeric receptor of the invention comprises amino acids 1447 to 2424 of the sequence shown in FIG. 8, or the corresponding sequence of Notch receptor other than Notch 1. In a further particularly preferred embodiment, a chimeric receptor of the invention comprises amino acids 1447 to 2555 of the sequence shown in FIG. 8, or the corresponding sequence of Notch receptor other than Notch 1. In a further particularly preferred embodiment, a chimeric receptor of the invention comprises amino acids 1396 to 2424 of the sequence shown in FIG. 8, or the corresponding sequence of Notch receptor other than Notch 1. In a further particularly preferred embodiment, a chimeric receptor of the invention comprises amino acids 1396 to 2555 of the sequence shown in FIG. 8, or the corresponding sequence of Notch receptor other than Notch 1. It is preferred that the chimeric receptor comprises said sequences of Notch1, and thus of the sequence shown in FIG. 8.

The term “heterologous ligand-binding domain” as used herein refers to a domain other than the ligand-binding domain of a Notch receptor, i.e. a domain other than the extracellular-ligand binding domain of Notch1, Notch2, Notch3 or Notch4. The heterologous ligand-binding domain can be any domain that can be bound by a ligand of choice. In particular, the ligand-binding domain can be the binding partner of any cell surface antigen or any soluble ligand. The versatility in the heterologous ligand-binding domain allows to select an appropriate ligand for any specific application. This way, activation of Notch signaling by the chimeric receptor of the invention can be induced at a selected time, a selected location/cell type, or both. Preferred examples of suitable extracellular ligand-binding domains are a ligand binding domain specific for a soluble ligand, a ligand binding domain specific for a cell surface antigen and a combination thereof. More preferred examples are:
an antibody or antigen binding part of an antibody, such as a single chain variable fragment (scFv), specific for a cell surface antigen;
an antibody or antigen binding part of an antibody, such as a single chain variable fragment (scFv), specific for an epitope in an antibody, a Fab fragment, a F(ab)2 fragment directed against a cell surface antigen;
an extracellular Fe-binding domain of an Fc receptor or a ligand-binding fragment thereof,
an extracellular domain that comprises an epitope for an antibody that can crosslink the chimeric receptor without involvement of a surface molecule;
an extracellular domain that comprises a moiety, such as biotin, that can be crosslinked by an agent with multiple binding sites for that moiety, such as streptavidin (resulting in clustering of multiple chimeric receptors upon addition of said agent).

In principle the following types of surface antigens can be used in accordance with the invention:
1. tumor specific antigens;
2. antigens that have a higher level of expression on tumor cells as compared to the expression level on non-tumor cells;
3. antigens that are expressed on both tumor cells and non-tumor cells, but where activation of T cells expressing the chimeric receptor of the invention induced by non-tumor cells results in side-effects that are acceptable, such as CD19 and CD20;
4. antigens that are expressed on both tumor cells and non-tumor cells, but that are specific for tumor cells in combination with one or more other antigens, such as a T cell epitope; and
5. antigens expressed on cells surrounding a tumor, such as PDL1 and PDL2.

In a preferred embodiment, a cell surface antigen is a tumor antigen and the heterologous extracellular ligand-binding domain is an antibody or antigen binding part of an antibody specific for said tumor antigen. Preferred examples of tumor antigens are TAG-72, calcium-activated chloride channel 2, 9D7, Ep-CAM, EphA3, Her2/neu, mesothelin, SAP-1, BAGE family, MC1R, prostate-specific antigen, CML66, TGF-βRII, MUC1, CD5, CD19, CD20, CD30, CD33, CD47, CD52, CD152 (CTLA-4), CD274 (PD-L1), CD273 (PD-L2) CD340 (ErbB-2), GD2, TPBG, CA-125, MUC1, immature laminin receptor and ErbB-1.

A skilled person is well capable of identifying soluble ligand and their binding partners that can be used in a chimeric antigen receptor according to the invention. Examples of suitable soluble ligands are antibodies directed against an epitope in the extracellular domain of the chimeric Notch receptor or molecules such as streptavidin in combination with biotinylated extracellular domains of the chimeric Notch receptor. A combination of a ligand binding domain specific for a soluble ligand and a ligand binding domain specific for a cell surface antigen is also possible. In that case Notch signaling will only be induced if both the soluble ligand and the cell surface antigen are present. For instance, an ectodomain can consist of an antibody to a peptide neo-epitope or to a Biotin or FITC moiety that is itself incorporated in another antibody (a “switch” antibody) directed to a surface antigen on a tumor. As a consequence, activation of the Chimeric Notch receptor will only occur if, in addition to the cell surface antigen targeted by the switch antibody, the switch antibody itself is also present. This set up is described in Ma et al 2016, which is incorporated herein by reference, and permits temporary control of the receptor (turning it on and off only when desired) as well as quantitative control (by in- or decreasing the concentration of the switch antibody.

The chimeric receptor of the invention further optionally comprises a linking sequence located between the transmembrane domain and the heterologous extracellular ligand-binding domain. Such linking sequence preferably comprises up to 30 amino acids, such as 2, 3, 4, 5, 6, 7, 8, 9 or 10 amino acids.

The percentage of identity of an amino acid sequence or nucleic acid sequence, or the term “% sequence identity”, is defined herein as the percentage of residues of the full length of an amino acid sequence or nucleic acid sequence that is identical with the residues in a reference amino acid sequence or nucleic acid sequence after aligning the two sequences and introducing gaps, if necessary, to achieve the maximum percent identity. Methods and computer programs for the alignment are well known in the art, for example “Align 2”.

In amino acid sequences as depicted herein amino acids are denoted by single-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine.

As used herein the terms “specific for” and “specifically binds” or “capable of specifically binding” refer to the non-covalent interaction between a ligand and a ligand-binding domain, such as an antibody or an antigen binding part thereof and its antigen or a soluble ligand and its binding partner. It indicates that the ligand preferentially binds to said ligand-binding domain over other domains.

An “antigen binding part of an antibody” is defined herein as a part of an antibody that is capable of specifically binding the same antigen as the antibody, although not necessarily to the same extent. The part does not necessarily need to be present as such in the antibody and includes different fragments of the antibody that together are capable of binding the antigen, such as a single-chain variable fragment (ScFv), a fusion protein of the variable regions of the heavy and light chains of an antibody.

A “cell surface antigen” as used herein refers to an antigen or molecule that is expressed at the extracellular surface of a cell.

As used herein “tumor antigen” refers to an antigen expressed on cells of a tumor. A tumor antigen is also referred to as a tumor-associated antigen (TAA).

A “soluble ligand” as used herein refers to a water-soluble ligand for which a binding partner can be used as extracellular domain of the chimeric receptor of the invention. It is preferred that the soluble ligand can be administered to a subject, e.g. by injection, such as intravenous injection, or orally.

Also provided is a nucleic acid molecule comprising a sequence encoding a chimeric receptor according to the invention. Also provided is a vector comprising the nucleic acid molecule according to the invention. In a preferred embodiment, the vector is a viral vector, e.g., a lentiviral vector or a retroviral vector. In another preferred embodiment, the vector comprises or is a transposon. Said nucleic acid molecule or vector may additionally comprise other components, such as means for high expression levels such as strong promoters, for example of viral origin, that direct expression in the specific cell in which the vector is introduced, and signal sequences. In a preferred embodiment, the nucleic acid molecule or vector comprises one or more of the following components: a promoter that drives expression in T cells, such as the EF1a promoter or the 5′ LTR of MSCV, a C-terminal signal peptide such as from the GMCSF protein or the CD8 protein for targeting to the plasma membrane and a polyadenylation signal.

Also provided is an isolated cell, comprising the nucleic acid molecule or vector according to the invention. The isolated cell is preferably an immune cell, such as natural killer cell, macrophage, neutrophil, eosinophil, or T cell. The nucleic acid molecule or vector may be introduced into the cell, preferably immune cells, by any method known in the art, such as by lentiviral transduction, retroviral transduction, DNA electroporation, or RNA electroporation. The nucleic acid molecule or vector is either transiently, or, preferably, stably provided to the cell. Methods for transduction or electroporation of cells with a nucleic acid are known to the skilled person.

In general, the chimeric receptors of the invention are advantageously used to improve T cell function and/or T cell survival, preferably of T cells reactive against tumors. Provided is therefore a method for improving T cell function and/or T cell survival in a subject in need thereof, the method comprising administering to the subject a therapeutically effective amount of a chimeric receptor, a nucleic acid molecule, a vector or a cell, preferably a T cell, according to the invention. Improving T cell function and/or T cell survival preferably comprises preventing or inhibiting T cell exhaustion. In a preferred aspect the subject is suffering from cancer. Said cell is preferably a T cell, preferably an autologous T cell of a subject suffering from cancer, such as a tumor derived T cell or a tumor infiltrating lymphocyte (TIL) or a T cell isolated from blood of the subject.

Also provided is a chimeric receptor, nucleic acid molecule or vector according to the invention, or a cell comprising the nucleic acid molecule or vector according to the invention for use in therapy. Preferably, said therapy is immunotherapy, more preferably tumor immunotherapy. In a preferred embodiment said tumor immunotherapy comprises adoptive cell transfer, more preferably adoptive T cell transfer.

Also provided is therefore a method for immunotherapy in a subject in need thereof, the method comprising administering to the subject a therapeutically effective amount of a chimeric receptor, a nucleic acid molecule, a vector or a cell according to the invention. In a preferred embodiment, such method comprises administration of a cell or population of cells according to the invention.

“Adoptive cell transfer” refers to the transfer of cells into a patient. In particular, “adoptive T cell transfer” refers to the transfer of T cells into a patient. The cells may have originated from the patient itself or may have come from another individual. Adoptive T cell transfer preferably comprises transfer of tumor infiltrating lymphocytes (TILs) or T cells isolated from blood, preferably derived from the subject or patient to be treated. If T cells isolated from blood are used, the T cells further preferably express a chimeric antigen receptor (CAR) or tumor specific T cell receptor.

“TILs” refers to autologous T cells found in or around the tumor of the patient to be treated. The T cells are expanded in vitro, e.g. cultured with cytokines such as interleukin-2 (IL-2) and anti-CD3 antibodies, and transferred back into the patient. Upon administration in uluo, TILs reinfiltrate the tumor and target tumor cells. Prior to TIL treatment, patients can be given nonmyeloablative chemotherapy to deplete native lymphocytes that can suppress tumor killing. Once lymphodepletion is completed, patients are then infused with TILs, optionally in combination with IL-2. Procedures for immunotherapy with adoptive T cell transfer including TILs, are well known in the art. In a preferred embodiment, TILs used in accordance with the invention are provided with a nucleic acid molecule or vector according to the invention after isolation from the patient. It is further preferred that the TILs express a chimeric receptor according to the invention.

“Immunotherapy” as used herein refers to treatment of an individual suffering from a disease or disorder by inducing or enhancing an immune response in said individual. Tumor immunotherapy relates to inducing or enhancing an individual's immune response against a tumor and/or cells of said tumor. Immunotherapy according to the invention can be either for treatment or prevention. “Treatment” means that the immune response induced or enhanced by the immunotherapy component ameliorates or inhibits an existing tumor. “Prevention” means that the immunotherapy component induces a protective immune response that protects an individual against developing cancer.

Also provided is a method of treating cancer in a subject in need thereof, the method comprising administering to the subject an effective amount of T cells comprising a nucleic acid sequence encoding the chimeric receptor according to the invention. Said T cells are preferably autologous T cells, such as TILs or T cell isolated from blood of the subject.

Tumors that can be treated or prevented using therapy based on a chimeric receptor according to the invention and/or a cell, preferably T cell, more preferably autologous T cells, such as TILs or T cells isolated from blood, provided with a nucleic acid molecule encoding a chimeric antigen receptor according to the invention or expressing a chimeric antigen receptor according to the invention can be any type of tumor, including primary tumors, secondary tumors, advanced tumors and metastases. Non-limiting examples tumors that can be treated or prevented in accordance with the invention are acute myeloid leukemia (AML), chronic myeloid leukemia (CML), chronic lymphocytic leukemia (CLL), acute lymphoblastic leukemia (ALL), chronic myelomonocytic leukemia (CMML), lymphoma, multiple myeloma, eosinophilic leukemia, hairy cell leukemia, Hodgkin lymphoma, non-Hodgkin lymphoma, large cell immunoblastic lymphoma, plasmacytoma, lung tumors, small cell lung carcinoma, non-small cell lung carcinoma, pancreatic tumors, breast tumors, liver tumors, brain tumors, skin tumors, bone tumors, colon tumors, rectal tumors, anal tumors, tumors of the small intestine, stomach tumors, gliomas, endocrine system tumors, thyroid tumors, esophageal tumors, gastric tumors, uterine tumors, urinary tract tumors and urinary bladder tumors, kidney tumors, renal cell carcinoma, prostate tumors, gall bladder tumors, tumors of the head or neck, ovarian tumors, cervical tumors, glioblastoma, melanoma, chondrosarcoma, fibrosarcoma, endometrial, esophageal, eye or gastrointestinal stromal tumors, liposarcoma, nasopharyngeal, thyroid, vaginal and vulvar tumors.

A “subject” as used herein is preferably a mammal, more preferably a human.

“T cells” or “TILs” referred to herein can be either CDT′ or CDR′ T cells or TILs or a combination of CD4⁺ or CD8⁺ T cells or TILs. In a preferred embodiment T cell or TILs are CD8⁺ T cells or TILs.

The invention also provides a genetically modified T cell, which is transduced by the nucleic acid molecule or vector of the invention. Said modified T cell is preferably a tumor derived T cell or a tumor infiltrating lymphocyte (TIL). Further, an isolated cell according to the invention is preferably a T cell, more preferably a tumor derived T cell or a TIL. In a particularly preferred embodiment, the T cell is an autologous T cell isolated from a patient suffering from cancer, i.e. an autologous TIL or an autologous T cell isolated from blood. It is further preferred that the T cell expresses a chimeric antigen receptor according to the invention.

In on aspect, treatment based on a chimeric receptor according to the invention is combined with at least one further immunotherapy component. Such further immunotherapy component can be any immunotherapy component known in the art. Preferably, said further immunotherapy component is selected from the group consisting of cellular immunotherapy, antibody therapy, cytokine therapy, vaccination and/or small molecule immunotherapy, or combinations thereof.

In a preferred embodiment, treatment with a chimeric receptor is combined with antibody-based immunotherapy, preferably comprising treatment using antibodies directed against a co-inhibitory T cell molecule. Co-inhibitory T cell molecules are also referred to as immune checkpoints. Preferred examples of co-inhibitory T cell molecules are cytotoxic T-lymphocyte antigen-4 (CTLA-4), programmed death-1 (PD-1), PD-ligand 1 (PD-L1), PD-L2, Signal-regulatory protein alpha (SIRPα), T-cell immunoglobulin- and mucin domain-3-containing molecule 3 (TIM3), lymphocyte-activation gene 3 (LAG3), killer cell immunoglobulin-like receptor (KIR), CD276, CD272, A2AR, VISTA and indoleamine 2,3 dioxygenase (IDO). An antibody against a co-inhibitory T cell molecule that is combined with a chimeric receptor or cell comprising a chimeric receptor according to the invention is therefore preferably selected from the group consisting of an anti-CTLA4 antibody, an anti-PD-1 antibody, an anti-PD-L1 antibody, an anti-PD-L2 antibody, an anti-SIRPα antibody, an anti-TIM3 antibody, an anti-LAG3 antibody, an anti-CD276 antibody, an anti-CD272 antibody, an anti-KIR antibody, an anti-A2AR antibody, an anti-VISTA antibody, anti TWIT antibody and an anti-IDO antibody. Suitable antibodies used as a further immunotherapy component are nivolumab, pembrolizumab, lambrolizumab, ipilimumab and lirilumab.

As demonstrated in the Examples, Notch signaling decreases expression of co-inhibitory T cell molecules. Also provided is therefore a method for enhancing efficacy of an antibody-based immunotherapy as defined herein in a subject suffering from cancer and being treated with said antibody, the method comprising administering to the subject a therapeutically effective amount of T cells expressing the chimeric receptor according to the invention. Said T cells are preferably autologous T cells, such as autologous TILs or T cells isolated from blood of the subject.

In a further preferred embodiment, treatment with a chimeric receptor is combined with treatment involving a chimeric antigen receptor (CAR) or tumor specific T cell receptor. Preferably cells comprising and/or expressing a chimeric receptor according to the invention that further comprise a chimeric antigen receptor (CAR) are used. This is in particular preferred if T cells other than TILs, such as autologous T cells isolated from blood, are used. CARs are antigen-targeted receptors composed of intracellular T-cell signaling domains fused to extracellular tumor-binding moieties, mostly single-chain variable fragments (scFvs) from monoclonal antibodies. CARs specifically recognize (tumor) cell surface antigens, independent of MHC-mediated antigen presentation. CARs preferably contain an ectodomain (such as an antigen binding portion of an antibody) specific for a tumor associated antigen, coupled to a signaling domain, preferably of CD′g, and a costimulatory receptor, such as CD28 or 4-1BB. Said cells are preferably T cells, more preferably autologous T cells derived from the subject to be treated, such as from blood or the tumor.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Schematic of a Chimeric Antigen Receptor (CAR).

Shown is an scFv (single chain) ligand binding portion of an antibody, which is linked to the intracellular signaling domains of either the 4-1BB or the CD28 costimulatory receptor and to the CD3 zeta chain.

FIG. 2: Notch signaling pathway.

Shown light blue and in red are Jagged and Delta, two membrane bound ligands of Notch. The Notch receptor itself is depicted in orange. After ligand binding the intracellular domain of Notch (NICD) is cleaved off the membrane and translocates to the nucleus, where it forms a transcriptional activator in complex with CSL and MAML proteins.

FIG. 3: Notch deficiency leads to reduced effector functions in antiviral CD8 T cells. (A) Flow chart of experiment. Wild type (Notch1^{flox/flox}Notch2^{flox/flox}) or T cell specific Notch1/2 knock out mice (Notch1^{flox/flox}Notch2^{flox/flox}CD4-Cre) were infected intranasally with HkX31 influenza virus and after 10 days T cells (results shown from spleen) were isolated and stained for CD8 and binding to the D^{b}NP₃₆₆₋₃₇₄ MHC tetramer (B). (C) Number of D^{b}NP₃₆₆₋₃₇₄-specific CD8⁺ T cells in wild type (black bars) or Notch1/2KO mice (open bars). Percentage IFNγ (D) or Granzyme B producing cells (E-blue histogram-wild type; red histogram N1/2ko) among D^{b}NP₃₆₆₋₃₇₄-specific CD8⁺ T cells. (F) Relative mRNA levels for Granzyme B and Perforin in FACSorted D^{b}NP₃₆₆₋₃₇₄-specific CD8⁺ T cells. (G) HkX31 viral loads (H) mouse weight curves and (I) influenza-neutralizing antibody titers in blood of infected mice. All results from Backer et al. 2014.

FIG. 4: CD8 T cell-intrinsic requirement for Notch in generation of effective memory. Wild type or Notch1/2 knock out mice were first infected intranasally with HkX31 influenza virus and then reinfected after 43 days with PR8 influenza. (A) Percentages of D^{b}NP₃₆₆₋₃₇₄ MHC tetramer binding CD8⁺ T cells in blood 8 days after reinfection. (B) Numbers of D^{b}NP₃₆₆₋₃₇₄ MHC tetramer binding CD8⁺ T cells in spleens and lungs. (C) Rag1 deficient mice were reconstituted with CD45.1⁺ WT bone marrow (BM) mixed with CD45.2⁺ WT BM (black bars) or mixed with CD45.2⁺ Notch1/2KO BM (white bars). Mice were then infected and reinfected as in A. Shown on the left are responses of CD45.1⁺CD8⁺ T cells and on the right responses of CD45.2⁺CD8⁺ T cells. Also shown are responses of mice reconstituted with CD45.2⁺ KO BM only (grey bars). Results were normalized against the corresponding WT controls. (D) Percentage IFNγ, TNFα and Granzyme B producing CD8 T cells isolated from lungs and restimulated in vitro with NP₃₆₆₋₃₇₄ peptide and wild type splenic antigen presenting cells (note that the number of influenza specific T cells was similar in lungs-see FIG. 4B).

FIG. 5: Notch deficiency leads to reduced effector functions in antiviral CD8 T cells. (A). Gene Set Enrichment Analysis of differentially expressed genes (obtained by RNAseq) between influenza specific effector CD8 T cells from wild type or T cell specific Notch1/2 knock out mice. (B) mRNA levels for PD1 and Lag3 in wild type or Notch1/2ko effector T cells. (C) 10⁴ CD45.2 wild type or Notch1/2ko OT1 T cells were transferred into CD45.1 wild type congenic mice, which were subsequently infected with Ovalbumin NP₃₆₆₋₃₇₄ peptide expressing influenza.

Representative FACS histogram (left) and MFI (right) for PD1 on influenza-specific memory CD8 T cells in lungs 30 days after infection. (D) Flow chart for experiment: CD45.2 OT1 T cells were transduced with empty vector or NICD (Notch intracellular domain) encoding retroviral vector and transferred into CD45.1 wild type mice infected as in (C). After 7 days, T cells were isolated and analyzed by FACS for PD1 levels (E).

FIG. 6: Physiological Notch responses are very sensitive to NICD. (A) Activation of the Notch responsive HES1-luciferase reporter induced by different levels of nuclear release of mER-NICD1 or constitutive NICD1 expression. U2OS cells were transfected with reporter plasmids expressing Firefly luciferase, a plasmid constitutively expressing Renilla luciferase and an empty vector control, mER-NICD or NICD1, respectively. Tamoxifen (4-HT) was added at the indicated concentrations. Firefly luciferase activities were normalized to Renilla luciferase activities from the same samples and are displayed as fold of empty vector control samples (mean+SD). Note that MER-NICD elicits 15.2-fold leaky induction in the absence of 4-HT. (B, C) Flow cytometric analysis of thymocytes after 2 weeks of co-culture on control OP9 cells. CD34⁺CD1a⁻ progenitors were transduced with NICD1, mERNICD1 or an empty vector control prior to co-culture. Tamoxifen was added to mER-NICD1 and empty vector transduced cultures at the concentrations indicated. (B) Transduced cells were analyzed for surface expression of CD4 and CD8 to assess T cell differentiation. (C) ILC2 differentiation as determined by expression of CRTH2 on transduced lineage-cells.

FIG. 7: The anti-TA-chNotch receptor. The LNR, heterodimerization, transmembrane and intracellular domains of Notch are fused to an antibody neo-ectodomain directed against a surface molecule on an adjacent cell, such as a tumor antigen (TA). Binding of the antibody neo-ectodomain to a ligand on an opposing cell, such as a tumor cell, will induce cleavage by TACE and γ-secretase, resulting in translocation of NICD to the nucleus and transactivation of endogenous Notch target genes. The anti-TA-chNotch receptor is inactive in the absence of the activating surface antigen.

FIG. 8: Amino acid sequence of Notch1 receptor. Sequence of UniProtKB/Swiss-Prot: P46531.4.

FIG. 9. Notch can protect CD8 T cells from developing hallmarks of exhaustion.

(A) OT-1 CD8⁺ T cells were activated and transduced with viruses expressing EV or NICD coupled to IRES-Thy1.1 and rested for 5 days. Subsequently, cells were co-cultured overnight with B16-F10 melanoma cells (not expressing Ovalbumin) and then stained for Thy1.1 (to identify transduced cells) and Granzyme B and analyzed by flow cytometry. Note that Thy1.1⁻ cells were gated out of the analysis. Note furthermore that the expression level of Thy1.1 differs between EV and the NICD construct due to the size of the NICD insert. (B) OT-1 T cells were activated and transduced as in (A). Five days after transduction, cells were cultured for an additional 6 days and fresh B16-F10 melanoma cells expressing Ovalbumin (B16-Ova) were added daily for repeated TCR stimulation leading to exhaustion. Cells were then stained for Thy1.1 and PD1 and analyzed by flow cytometry. (C) OT-1 CD8⁺ T cells were treated as in (B) and the percentages of Thy1.1⁺ cells were analyzed by flow cytometry after different times of coculture with B16-Ova, as indicated in the figure. (D) OT-1 CD8⁺ T cells were activated and transduced with viruses expressing EV or mER-NICD (a tamoxifen inducible version of NICD) and cultured with B16-Ova as in (C) without or with 0.05 mM (+) or 0.5 mM (++) tamoxifen. Thy1.1⁺ cells were then analyzed by flow cytometry for IFNg, IL10, Granzyme B and PD1 expression.

FIG. 10: Generation and expression of a chimeric Notch receptor (CNR) directed against CD19. (A) schematic of experiment. The CNR contains an extracellular ScFv domain specific for human CD19. A human CD19 protein, fused to a human IgG1 IFc portion, was used to detect surface expression of the CNR. A fluorescently labeled anti-human antibody was then used to detect the hCD19-Ig fusion protein. PEST=Notch PEST domain; AF647=Alexa Fluor 647. (B) HEK293T cells were transfected with a CNR expression construct or control and subsequently stained without or with different concentrations of hCD19-Ig, followed by fluorescently labeled anti-human antibody.

### EXAMPLES

### Example 1

### Results

To examine the role of Notch in CD8 T cell responses, in Backer et al. 2014 mice carrying T cell-specific deletions in the Notch1 and Notch2 genes (Notch1/2ko) were infected with influenza virus. At the peak of the response, influenza-specific CD8 T cells were detected using D^{b} tetramers loaded with an immuno-dominant peptide of influenza (FIG. 3a,b). Although the magnitude of the influenza-specific CD8 T cell response was similar in wild type (WT) and Notch1/2ko mice (FIG. 3C and not shown), Notch1/2 deficient T cells produced less IFNγ and Granzyme B than WT CD8 T cells (FIG. 3d,e,f). Notch1/2ko mice were also less able to clear the influenza virus and exhibited delayed recovery (FIG. 3g,h). Titers of neutralizing antibodies were, if anything, elevated in Notch1/2ko mice (Figure Si), suggesting that their inability to clear the virus was caused by their ineffective CD8 T cell response.

Memory responses to influenza were affected even more severely by Notch1/2 deficiency in all anatomical locations examined (FIG. 4a,b). Defective memory activity was a consequence of a CD8 T cell-intrinsic function of Notch, as shown by the inability of Notch1/2ko CD8 T cells to expand even in mixed bone marrow chimeras (FIG. 4c). Surprisingly, normal numbers of Notch1/2ko memory CD8 T cells were found in lungs (FIG. 4b), but these hardly produced effector molecules (FIG. 4d).

The profound unresponsiveness of Notch1/2ko CD8 T cells is reminiscent of “exhaustion”: inability to fully respond due to expression of inhibitory receptors, such as PD1 and Lag3 (Wherry and Kurachi, 2015). This notion was reinforced by whole transcriptome analysis of Notch1/2ko CD8 effector T cells. Among differentially expressed genes between Notch1/2ko and WT effector T cells, the most significantly enriched gene set was derived from a comparison between acute and chronic infection with LCMV (FIG. 5a), the prototypical model used to study T cell exhaustion (Wherry and Kurachi, 2015). Indeed, mRNA levels for both PD1 and Lag3 were elevated in Notch1/2ko CD8 effector T cells (FIG. 5b).

Importantly, expression of PD1 was elevated on the surface of Notch1/2 deficient OT1 T cells transferred into WT congenic recipient mice that were infected with Influenza-Ova (to which the OT1 T cell receptor responds) (FIG. 5c). The endogenous repertoire of T and B cells effectively clears influenza virus in these mice, excluding viral persistence as an explanation for the elevated PD1 expression selectively on Notch1/2ko T cells. Furthermore, expression of an activated Notch1 allele (NICD) specifically in Notch1/2ko OT1 T cells strongly suppressed PD1 expression (FIG. 5e). This demonstrates that Notch suppresses expression of PD1 in a CD8 T cell-intrinsic manner.

Expression of the intracellular domain of Notch (NICD) mimics activation of Notch, both in CD4 T cells and CD8 T cells (Helbig et al. 2012; Backer et al. 2014; Amsen et al. 2007). Notch signaling is exquisitely sensitive and the number of nuclear NICD molecules obtained by overexpression of an NICD construct likely vastly exceeds the number of molecules obtained after ligand-mediated activation. This is illustrated by experiments using tamoxifen inducible MER-NICD alleles in thymic progenitor cells. Culturing CD34⁺CD1a⁻ human thymic progenitor cells on OP9 stromal cells only resulted in differentiation if NICD was expressed (FIG. 6b). Strikingly, maximal differentiation of CD4⁺CD8⁺ double positive cells was already obtained by the leaky activity of MER-NICD in the absence of tamoxifen (FIG. 6b), conditions that result in very weak transactivation of a luciferase reporter construct (FIG. 6a). Furthermore, increasing activity of MER-NICD by addition of tamoxifen resulted in a gradual conversion of differentiation from double positive thymocytes into CRTH2⁺ ILC2 cells (FIG. 6c). These results emphasize the exquisite sensitivity of endogenous response programs to NICD. Furthermore, they show that the strength of Notch signaling sometimes qualitatively affects the biological response to this receptor. (These results have been published in Gentek et al. 2013)

### Materials and Methods

Mice. All mice were on a C57BL/6 background. Notch1^{flox/flox}Notch2^{flox/flox} Cd4-Cre mice were used (Amsen et al. 2014; Amsen et al. 2004). Cre-negative littermates were used in all experiments. Transgenic mice expressing the OT-I TCR (003831) are available from Jackson Laboratories. Mice were bred and housed in specific pathogen-free conditions at the Animal Centers of the Academic Medical Center (AMC, Amsterdam, The Netherlands). Mice (both male and female) were between 8-16 weeks of age at the start of the experiment. During infection experiments, wild-type and Notch1-2-KO mice were housed together to avoid cage bias. No intentional method for randomization was used. No formal method for blinding was used, except for determination of viral loads and hemagglutination assay, where the operator did not know mouse genotypes. Mixed-bone marrow (BM) chimeras containing wild-type and Notch1-2-KO BM at a 1:1 ratio were generated via intravenous injection of 5-10×10⁶ donor BM cells into lethally irradiated RAG1-deficient mice. Wild-type and Notch1-2-KO cells of donor origin were identified with the congenic CD45.1/2 markers. BM chimeras were used at 12 weeks after engraftment. All mice were used in accordance of institutional and national animal experimentation guidelines. All procedures were approved by the local Animal Ethics Committees.

Media, reagents and mAbs for mouse studies. Culture medium was Iscove's modified Dulbecco's medium (IMDM; Lonza) supplemented with 10% heat-inactivated FCS (Lonza), 200 U/ml penicillin, 200 μg/ml streptomycin (Gibco), GlutaMAX (Gibco) and 50 μM β-mercaptoethanol (Invitrogen) (IMDMc). All directly conjugated monoclonal antibodies used for flow cytometry were purchased from eBioscience, San Diego, Calif., unless stated otherwise: anti-CD3ε (clone 145-2C11), anti-CD4 (clone GK1.5), anti-CD8α (Ly-2, clone 53-6.7), anti-CD28 (clone 37.51), anti-CD44 (clone IM7), anti-CD45.1 (clone A20, BD Biosciences), anti-CD45.2 (clone 104), anti-CD127 (anti-IL7Rα, clone A7R34), anti-Granzyme B (clone GB-11, Sanquin PeliCluster), anti-IL-2 (clone JES6-5H4), anti-IFN-γ (clone XMG1.2), anti-KLRG-1 (clone 2F1), and anti-TNFα (clone MP6-XT22), isotype control (cat. #3900S) (Cell Signaling Technology).

Influenza infection. Mice were intranasally infected with 100-200×50% tissue culture effective dose (TCID₅₀) of the H3N2 influenza A virus HKx31 (Belz et al. 2000), influenza A/WSN/33, A/WSN/33-OVA(I) (Topham et al. 2001), A/PR/8/34 (HIN1) or the recombinant A/PR/8/34 expressing the LCMV gp.41 epitope (Mueller et al. 2010). Stocks and viral titers were obtained by infecting MDCK or LLC-MK2 cells as described previously (Van der Sluijs et al. 2004). At indicated time intervals, blood samples were drawn from the tail vein or mice were sacrificed and organs were collected to determine numbers of influenza-specific CD8⁺ T cells. Influenza-specific CD8⁺ T cells were enumerated using anti-CD8 (53-6.7) and PE- or APC-conjugated tetramers of H-2D^{b} containing the influenza-A-derived nucleocapsid protein (NP) peptide NP₃₆₆₋₃₇₄ ASNENMETM (produced at the Sanquin Laboratory for Blood Research). A/PR/8/34 viral loads in lungs of infected mice were determined by isolating lung mRNA and detection of viral mRNA by quantitative PCR using the following primers and probe specific for the A/PR/8/34 M gene. Sense primer: 5′-CAAAGCGTCTACGCTGCAGTCC-3′; antisense primer: 5′-TTTGTGTTCACGCTCACCGTGCC-3′; Probe: 5′-AAGACCAATCCTGTCACCTCTGA-3′.

Sera were tested for the presence of neutralizing antibodies to this virus by hemagglutination inhibition (HI) assay as described previously using four hemagglutinating units of virus and turkey erythrocytes (Palmer et al. 1975). Values represent the maximum serum dilution at which agglutination was completely inhibited.

Flow cytometry and cell sorting. For intracellular cytokine and granzyme B staining, splenocytes and total lung samples were stimulated with 1 μg/ml of the MHC class I restricted influenza-derived peptide NPaw74 ASNENMETM for 4 h in the presence of 10 μg/ml brefeldin A (Sigma) to prevent cytokine release. Cells were stained with the relevant fluorochrome-conjugated mAbs for 30 min at 4° C. in PBS containing 0.5% BSA and 0.02% NaN. For intracellular staining, cells were fixed and permeabilized using the Cytofix/Cytoperm (BD Biosciences). Data acquisition and analysis was done on a FACSCanto (Becton Dickinson) and FlowJo software. To isolate H-2 Db-NP tetramer-positive CD8⁺ T cells from influenza infected mice, single cell suspensions of spleens were stained with influenza-specific tetramers and various markers. Cells were sorted using FACSAria cell sorters (BD Biosciences).

For analysis of human thymoctes, distinction of live and dead cells was based on staining with 7-Aminoactinomycin D (7-AAD, eBiosciences) or fixable live/dead dyes (Invitrogen). Data were acquired on a LSR Fortessa flow cytometer (BD Bioscience) and analyzed using FlowJo software (TreeStar). Single cell suspensions were stained with antibodies directly labeled with Fluorescein Isothiocyanate (FITC), Phycoerythrin (PE), Phycoerythrin-Cyanine 5 (PE-Cy5), PE-Cy5.5, PE-Cy7, PerCP-Cy5.5, Allophycocyanin (APC)/Alexa Fluor 647, APC-Cy7, AF700 (all BD Bioscience, Biolegend or MACS Miltenyi), Horizon V500 (HV500, BD Bioscience), Brilliant Violet 421 (BV421), BV711 and BV785 (all Biolegend). Antibodies specific for the following human antigens were used: CD1a, CD3, CD4, CD7, CD8, CD11c, CD14, CD19, CD25, CD34, CD45, CD56, CD94, CD117 (cKit), CD123, CD127 (IL-7Rα), CD161, CD294 (CRTH2), CD303 (BDCA2), CD336 (Nkp44). CD278 (ICOS), TCRαβ. TCRγδ and FcER1. Anti-mouse CD90.1 (Thy1.1)-FITC, -PE or -APC-eFluor 780 (eBioscience) were used to detect cells transduced with MSCV—IRES-Thy1.1 retroviruses.

Retroviral transductions and adoptive transfers of mouse CD8⁺ T cells. Virus was produced in PlatE cells as described (Amsen et al. 2004). Total splenocytes from CD45.2⁺ OT-I wild-type or OT-I Notch1-2-KO mice were incubated with 1 nM OVA₂₅₇₋₂₆₄ peptide, and next day cells were spin-infected (700×g for 90 min at 37° C.) with viral supernatant (with 8 μg/ml polybrene), followed by 5 h at 37° C. Medium was replaced and next day, live T cells were isolated by density centrifugation (Lymphoprep, Axis-shield PoC) and between 7.5×10² and 5×10⁴ cells were transferred into timed influenza-OVA infected CD45.1⁺ mice. Donor OT-1 T cells were detected 5-10 days after transfer as CD45.2⁺CD8⁺ and Thy1.1 or GFP triple positive cells.

Virus production and transduction of human thymocytes. For virus production, Phoenix GALV packaging cells were transiently transfected using FuGene HD (Promega). Virus containing supernatant was harvested 48 h after transfection, snap frozen on dry ice and stored at −80° C. until use. For transduction, cells were incubated with virus supernatant in plates coated with Retronectin (Takara Biomedicals) for 6-8 h at 37° C. the following day.

Retroviral constructs used for human thymocyte experiments. The human NICD1-IRES-Thy1.1-MSCV construct has been described before (Amsen et al. 2004). To generate the mER-NICD fusion, an N-terminal mER domain was PCR amplified using the following primers: GATCAGGAATTCCACACCATGGGAGATCCACGAAATGAA and GATCAGGATATCCACCTTCCTCTTCTTCTTGG and cloned into the EcOR1 and EcORV sites of pBluescript (pBS) to create mER-pBS. Human NICD1 lacking a translation initiation signal was PCR amplified using these primers: ATCGGAGGTTCTCGCAAGCGCCGGCGGCAGCAT and GATCAGAAGCTTGAATTCTTACTTGAAGGCCTCCGGAATG and subsequently cloned into the EcORV and HindIII sites of mER-pBS. The mER-NICD1 fusion insert was then cloned into IRES-Thy1.1-MSCV using BamH1 and Cla1.

Gene expression profiling mouse studies. H-2 D^{b}-NP₃₆₆₋₃₇₄⁺CD8⁺ T cells were isolated from spleens of influenza infected mice by flow cytometry. Total RNA was extracted with TRIzol reagent (Invitrogen) according to the manufacturer's protocol. For Deep sequencing analysis, total RNA was further purified by nucleospin RNAII columns (Macherey-Nagel) and RNA was amplified using the Superscript RNA amplification system (Invitrogen) and labeled with the ULS system (Kreatech), using either Cy3 or Cy5 dyes (Amersham). Sequences were obtained by pooling 10 samples in one lane on a HiSeq2000 machine. Between 17 and 27 million reads were obtained per sample.

Read mapping (TopHat) and determining differentially expressed genes (DESeq) was done as described in (Anders et al. 2013). Reads were mapped against the mouse reference genome (build mm9) using TopHat (version 1.4.0), which allows to span exon-exon junctions. TopHat was supplied with a known set of gene models (NCBI build 37, version 64). In order to obtain per sample genecounts HTSeq-count was used. This tool generates genecounts for each gene that is present in the provided Gene Transfer Format (GTF) file. Genes that have zero counts across all samples were removed from the dataset. Statistical analysis was performed using the R package DESeq. Differentially expressed genes were determined between the SLEC and MPEC samples, and between the wild type and knock-out samples. DESeq assumes that gene counts can be modelled by a negative binomial distribution. For sample normalisation the ‘size factors’ were determined from the count data. The empirical dispersion was determined with the ‘pooled’ method, which used the samples from all conditions with replicates to estimate a single pooled dispersion value. Subsequently, a parametric fit determines the dispersion-mean relationship for the expression values resulting in two dispersion estimates for each gene (the empirical estimated, and the fitted value). Using the ‘maximum sharingMode’ we selected the maximum of these two values to be more conservative. Finally, p-values and FDR corrected p-values were calculated.

To highlight biological processes that are over-represented in the set of differentially expressed genes we used Bioconductor package GOseq (Young et al. 2010), which was developed for the analysis of RNA-seq data. First we selected all genes with an FDR<0.5 from the SLEC-MPEC and WT-KO comparisons. Subsequently, the GO ‘Biological Processes’ gene sets were used to determine over-represented processes. In addition we used the ‘C7’ gene set from the Molecular Signatures Database (MSigDB; http://www.broadinstitute.org/gsea), which is a collection of annotated gene sets. Gene set C7 comprises immunologic signatures composed of gene sets that represent cell types, states, and perturbations within the immune system. The signatures were generated by manual curation of published microarray studies in human and mouse immunology. This gene set was generated as part of the Human Immunology Project Consortium (HIPC; http://www.immuneprofiling.org). An in-house R script was developed to convert the C7 gene set into a format that could be used by GOseq.

Statistical analysis. Figures represent means and error bars denote standard error of the mean (s.e.m.). Standard Student's 1-tests (unpaired, two-tailed) was applied with GraphPadPrism software. If 3 or more groups were compared One-way ANOVA with Bonferroni correction was used. P<0.05 was considered statistically significant.

Isolation of human thymic hematopoietic progenitors. Postnatal thymic (PNT) tissue specimens were obtained from children undergoing open heart surgery (LUMC, Liden, the Netherlands); their use was approved by the AMC ethical committee in accordance with the declaration of Helsinki. Cell suspensions were prepared by mechanical disruption using the Stomacher 80 Biomaster (Seward). After overnight incubation at 4 C, thymocytes were isolated from a Ficoll-Hypaque (Lymphoprep; Nycomed Pharma) density gradient. Single cell suspensions were enriched for CD34⁺ cells by MACS (Miltenyi Biotec), stained with fluorescently labeled antibodies and subsequently FACS sorted on a FACS Aria (BD Bioscience) as CD34⁺CD1a⁻CD3⁻CD56⁻BDCA2⁻ or CD34⁺CD1a⁺CD3⁻CD56⁻ BDCA2⁻, respectively (referred to in this study as CD34⁺CD1a⁻ and CD34⁺CD1a⁺). Purity of the sorted populations was >99%.

In vitro differentiation of thymic progenitors. Sorted thymic progenitors were cultured overnight in Yssel's medium containing 5% normal human serum, SCF (20 ng/ml) and IL-7 (10 ng/ml, both PeproTech). OP9 cells were mitotically inactivated by irradiation with 30Grey and seeded at a density of 5×10³/cm² one day prior to initiation of co-cultures. After transduction, thymic progenitors were added to pre-seeded OP9 cells. Co-cultures were performed in MEMα (Invitrogen) with FCS (20% Fetal Clone I, Hyclone) and IL-7 (5 ng/ml). In some cases, Flt3l (5 ng/ml, PeproTech) was added to the medium. Cultures were refreshed every 3-4 days. Differentiation assays for innate lymphoid cells were typically analyzed after 1 week, unless stated otherwise. Cells were harvested by forceful pipetting and passed through 70 mm nylon mesh filters (Spectrum Labs).

Reporter gene assays. U2OS cells were transiently transfected using the FuGene HD transfection reagent (Promega). Cells were co-transfected with a NOTCH-responsive promoter and either NICD1—MSCV Th1.1, mER-NICD1—MSCV Th1.1 or an empty vector control. To correct for differences in transfection efficiency, the pRL-CMV control vector was co-transfected, from which Renilla luciferase is expressed constitutively. Transfections were performed in triplicate. Where applicable, 4-Hydroxy-Tamoxifen (Sigma) was added after overnight incubation to induce nuclear translocation of mER-NICD1. Cells were lysed 48 h post transfection and luciferase activity was measured using the Dual Luciferase Reporter Assay System (Promega) on a Synergy HT microplate reader (Syntek). Two different Notch responsive reporter constructs were used, which have been described previously (Nam et al. 2007).

The Chimeric Notch receptor (ChNR) system. To generate a Chimeric Notch receptor the extracellular domain of Notch except the heterodomerization domain is replaced by a heterologous ligand binding domain consisting of an scFv antibody domain fused to the heterodimerization domain of Notch. This receptor will be activated by binding to the cognate ligand of the scFv antibody on the surface of an adjacent cell, but will remain silent when this surface antigen is not present (FIG. 7). ChNR can be expressed in CD4 T cells via retroviral transduction or other methods. If such modified T cells are adoptively transferred into patients, Notch can specifically be turned on only in these T cells.

The ChNR will typically not by itself be sufficient to fully activate T cells. For that, additional T cell receptor signals (or mimics thereof) are required. For instance, T cells can be derived from primary tumors (Tumor infiltrating lymphocytes-TIL) after selection for tumor reactivity. Also, ChNR can be used in conjunction with recombinant T cell receptors against tumor antigens or in T cells engineered to express traditional chimeric antigen receptors (CAR).

Many variations of this basic concept are possible. As ectodomain any antibody that recognizes a surface antigen can in principle be used and any surface antigen expressed on the surface of tumor cells can in principle be targeted. Finally, even ectodomains activated by soluble ligands are an option. For instance, an ectodomain can consist of an antibody to a peptide neo-epitope (as described in Rodgers et al. 2016) or to a Biotin or FITC moiety (as described in Ma et al. 2016) that is itself incorporated in another antibody (a switch antibody) directed to a surface antigen on a tumor. As a consequence, activation of the Chimeric Notch receptor will only occur if, in addition to the cell surface antigen targeted by the switch antibody, the switch antibody itself is also present. This set up would permit temporary control of the receptor (turning it on and off only when desired) as well as quantitative control (by in- or decreasing the concentration of the switch antibody. In all these situations, however, liberation of the intracellular domain of Notch from the Chimeric Notch receptors remains the central goal.

The preparation of an exemplary Chimeric notch receptor is described in example 2.

### Example 2

### Results

T cell exhaustion occurs when T cells are chronically stimulated via their T cell receptor. The results in example 1 show that CD8 T cells responding to an infection with influenza virus are protected from activation of this exhaustion program by Notch. Influenza infection does not, however, normally cause chronic stimulation of T cells. We therefore asked whether deliberate activation of Notch can also prevent exhaustion under conditions that normally do lead to exhaustion. To this end, we resorted to an in vitro system in which an activated Notch allele (NICD) can be introduced in T cells that are then subjected to repeated TCR stimulation. NICD was expressed in OT-1 CD8 T cells (which recognize the SIINFEKL peptide from the Ovalbumin protein in H2-K^{b}) using a retroviral expression system. An IRES-Thy1.1 sequence in this retroviral construct allows discrimination between the transduced T cells (Thy1.1⁺) and the untransduced T cells (Thy1.1-). Expression of NICD in CD8⁺ OT-1 T cells strongly enhanced effector functions, as evidenced for instance by the spontaneous production of the cytolytic effector protein Granzyme B (FIG. 9A). Transduced OT-1 cells were then repeatedly stimulated by daily addition of B16F10 melanoma cells expressing Ovalbumin (B16-Ova). These conditions result in prominent expression of the check-point molecule (and hallmark of exhaustion) PD1 on the surface of OT-1 T cells transduced with a control virus (Empty Vector-EV) (FIG. 9B, left). Expression of NICD, however, nearly completely prevented expression of PD1 (FIG. 9B, right). Expression of NICD also afforded a competitive advantage to the OT-1 T cells: the proportion of Th1.1⁺ cells in the population transduced with NICD gradually increased over time, whereas the Th1.1⁺ population remained stable when cells had been transduced with Empty Vector (FIG. 9C).

The concentration of active Notch molecules that is obtained after expression of the NICD allele is probably unphysiologically high. Moreover, it may not be possible to achieve similarly high levels of such active Notch molecules using the ChNR. To test whether the protective effects on CD8 T cells can also be obtained with weaker Notch stimulation, we made use of a Tamoxifen inducible version of NICD (also used in example 1, FIG. 6). This construct consists of NICD coupled at the N-terminus to the ligand binding domain of the Estrogen Receptor (ER), which has been mutated such that it responds only to Tamoxifen and no longer to Estrogen. This mutated ER domain (mER) sequesters NICD molecules in the cytoplasm by binding to heat shock proteins and thereby keeps it inactive. Upon addition of tamoxifen, the mER-NICD fusion protein however dissociates from these heat shock proteins, allowing NICD to become active. As shown by luciferase reporter assays (FIG. 6A), this fusion protein reaches much lower maximal levels of Notch activity than NICD itself and its activity can be controlled quantitatively by titration of Tamoxifen. Finally, this mER-NICD possesses some “leaky” Notch activity even in the absence of Tamoxifen, which is almost undetectable in luciferase reporter assays, yet can elicit physiological functions of Notch such as induction of differentiation of CD4⁺CD8⁺ thymocytes from thymic precursor cells (FIG. 6B). We therefore used this mER-NICD construct to examine the signal strength requirements for protection against exhaustion in CD8 T cells, again using the repetitive stimulation model with B16-Ova melanoma cells (as in A-C). Stimulation of mER-NICD with 0.5 or even 0.05 mM of tamoxifen indeed resulted in reduced expression of PD1 and production of the tolerogenic cytokine IL10 (FIG. 9D). It also mobilized production of effector molecules such as IFNg and Granzyme B. Remarkably, some of these effects were obtained even by the very low leaky NICD activity that is elicited by mER-NICD in the absence of tamoxifen. We thus conclude that Notch can protect CD8 T cells from developing hallmarks of exhaustion (expression of PD1, loss of production of effector molecules) even at relatively modest levels of Notch activity.

### Generation of Chimeric Notch Receptor

A chimeric Notch receptor consisting of an ScFv antibody domain directed against human CD19 was generated (ScFv as described in Molecular Immunology 1997; 34:1157-1165 and used in a CAR construct in J Immunother. 2009 September; 32(7): 689-702). This ScFv was fused in frame to the 5′end of the human NOTCH 1 protein truncated upstream of the extracellular heterodimerization domain (FIG. 10A).

Specifically, The GMCSF leader sequence (MLLLVTSLLL CELPHPAFLL) was fused in frame to the Igx light chain Variable domain followed by the Ig heavy chain Variable domain of FMC63-28Z anti CD19 ScFv (IPDIQMTQTTSSLSASLGDRVTISCRASQDISKYLNWYQQKPDGTVKLLIYHTSR LHSGVPSRFSGSGSGTDYSLTISNLEQEDIATYFCQQGNTLPYTFGGGTKLEITG STSGSGKPGSGEGSTKGEVKLQESGPGLVAPSQSLSVTCTVSGVSLPDYGVSWI RQPPRKGLEWLGVIWGSETTYYNSALKSRLTIIKDNSKSQVFLKMNSLQTDDTA IYYCAKHYYYGGSYAMDYWGQGTSVTVSSAAA), which was fused in frame with the C-terminus from the human full length NOTCH 1 protein starting at Isoleucine 1427 till Lysine 2555 (of the sequence as depicted in FIG. 8).

In an alternative construct, the C terminus of human NOTCH1 sequence used starts at Proline 1390. Both variants (beginning with Ile 1427 or Proline 1390, see sequence of FIG. 8) are made also with a deletion of the C-terminal PEST domain of human NOTCH 1 (ending at Alanine 2424 of the human NOTCH1 protein, see sequence of FIG. 8).

The fusion protein was then expressed from the pHEFTIG lentiviral expression vector (described in J Immunol 2009; 183:7645-7655 as “modified pCDH1”, and as “pHEF” in PNAS Aug. 9, 2011 108 (32) 13224-13229) after transfection into HEK293T cells and its presence at the cell surface was documented by staining with recombinant human CD19-Ig protein (FIG. 10B).

### Materials and Methods

Mice. Female or male OT-1 TCR transgenic mice (C57BL/6 strain) with transgenic inserts for TCRα-V2 and TCRβ-V5 genes that are specifically designed to target the ovalbumin residues 257-264 presented by H2-Kb, were bred and maintained in the animal facility of the Netherlands Cancer Institute (NKI, Amsterdam, The Netherlands). All animal experiments were performed according to protocols in compliance with institutional guidelines and approved by the Animal Ethics Committee of the NKI.

Cell lines and reagents. B16-F10 and B16-OVA tumor cell lines were cultured in Iscove's Modified Dulbecco's Medium (IMDM) with HEPES supplemented with 10% heat-inactivated Fetal Calf Serum (Bodingo BV), 5% L-glutamine (Lonza, Belgium) and 5% Penicillin/Streptomycin (Sigma, 10.000 U Penicillin and 10 mg Streptomycin). Platinum-Eco cells and HEK293T cells were cultured in Dulbecco's Modified Eagle Medium (DMEM) with HEPES supplemented with 10% heat-inactivated Fetal Calf Serum (Bodingo BV) and 5% L-glutamine (Lonza, Belgium). All cells were incubated at 37° C., 5% CO2.

Cell purification. A single cell suspension was obtained from the spleen and lymph nodes from OT-1 mice. CD8⁺ T cells were enriched and purified by Magnetic-Activated Cell Sorting (MACS). CD8α⁺ T cell Isolation Kit, mouse (Miltenyi Biotech) was used for the negative selection of CD8α⁺ T cells. The cells were then cultured up to two weeks with IMDM supplemented with 10% heat-inactivated Fetal Calf Serum (Bodingo BV), 5% L-glutamine (Lonza, Belgium), 5% Penicillin/Streptomycin (Sigma, 10.000 U Penicillin and 10 mg Streptomycin) and 50 μM β-mercapto-ethanol (Sigma Aldrich).

Retroviral transductions of murine CD8⁺ T cells. Retroviral stocks were generated by transfection of Platinum-Eco cells with the construct using FuGENE® HD reagent (Promega) according to the manufacturer's instructions. 3×10⁶ cells were plated in a 100 mm dish one day prior to transfection. 56 μl of FuGENE HD reagent was added to 879 μl of plasmid solution (0.020 μg/μl in OptiMEM (Gibco by Life Technologies)) and subsequently incubated for 10 minutes at RT. The complex solution was then added to the cells and incubated o/n at 37° C. Viral supernatant was collected and filtered with a 0.45 μM syringe filter to remove cell debris. Virus supernatants were made from pMSCV-EV and pMSCV-NICD. Retroviral vectors contained an IRES sequence enabling cap-independent translation and a Thy1.1 (CD90.1) selection marker, which was used for positive transduction selection. Activated CD8⁺ T cells purified from OT-1 mice were infected with virus with an addition of 10 μg/ml Polybrene (Merck) in a 24-well plate (1×10⁶ cells/well). The cells were spun at 2000 RPM for 90 min. at RT followed by incubation for 4 h at 37° C. and 5% CO2.

Transfection HEK293T cells. Cells were transfected with CNR-pHEFTIG or pHEFTIG empty vector in 6 well plate using Fugene HD reagent following manufacturer's instructions. After 48 hours, expression was analyzed by Flow Cytometry.

CD8⁺ T cell activation and re-stimulation. For efficient in vitro activation of the T cells, an engineered APC cell line MEC.B7.SigOVA (SAMBcd8⁺OK) that encodes the OVA257-264 (SIINFEKL) peptide was used. Following CD8⁺ T cell purification, 10⁶ CD8⁺ T cells were co-cultured with 105 SAMBOK cells in a 24-well plate for 24 hours. Cells were then collected and transduced. Cells were maintained at a cell density of ±1.5×10⁶ cells/ml until re-stimulation. Five days after transduction, 300.000 CD8⁺ T cells were co-cultured with 50.000 B16-F10/B16-OVA in a 96-flat bottom well plate (FIG. 5). T cells were removed from the adherent B16 cells and were seeded to new B16 cells every 24 hours. Four hours before each desired re-stimulation time point, Brefeldin A (1000×, Invitrogen, USA) was added. Cytokine production and expression of inhibitory receptors were assessed via flow cytometry.

Flow cytometry and antibodies. All samples were measured on the BD FACSymphony A5 (BD Biosciences). Prior to flow cytometry measurement, cells were stained extracellularly (in PBS containing 1.5% FCS at 4° C.) and were fixated and permeabilized using Cytofix/Cytoperm (BD Pharmingen). Cells were then stained intracellularly (in 1× PermWash at 4° C.). Human CD19 protein, fused to a human IgG1 Fc portion (R&D Systems), was used to detect surface expression of the CNR. A fluorescently labeled anti-human antibody (Invitrogen) was then used to detect the hCD19-Ig fusion protein.

### REFERENCES

Amsen, D., Blander, J. M., Lee, G. R., Tanigaki, K., Honjo, T., and Flavell, R. A. (2004) Instruction of distinct CD4 T helper cell fates by different notch ligands on antigen-presenting cells. Cell 117, 515-526
Amsen, D., Antov, A., Jankovic, D., Sher, A., Radtke, F., Souabni, A., Busslinger, M., McCright, B., Gridley, T., and Flavell, R. A. (2007) Direct regulation of Gata3 expression determines the T helper differentiation potential of Notch. Immunity 27, 89-99
Anders, S., McCarthy, D. J., Chen, Y., Okoniewski, M., Smyth, G. K., Huber, W., and Robinson, M. D. (2013) Count-based differential expression analysis of RNA sequencing data using R and Bioconductor. Nat Protoc 8, 1765-1786
Backer, R. A., Helbig, C., Gentek, R., Kent, A., Laidlaw, B. J., Dominguez, C. X., de Souza, Y. S., van Trierum, S. E., van Beek, R., Rimmelzwaan, G. F., ten Brinke, A., Willemsen, A. M., van Kampen, A. H., Kaech, S. M., Blander, J. M., van Gisbergen, K., and Amsen, D. (2014) A central role for Notch in effector CD8(+) T cell differentiation. Nat Immunol 15, 1143-1151
Belz, G. T., Xie, W., Altman, J. D., and Doherty, P. C. (2000) A previously unrecognized H-2D(b)-restricted peptide prominent in the primary influenza A virus-specific CD8(+) T-cell response is much less apparent following secondary challenge. J Virol 74, 3486-3493
Gentek R, Munneke J M, Helbig C, Blom B, Hazenberg M D, Spits H, Amsen D. Modulation of Signal Strength Switches Notch from an Inducer of T Cells to an Inducer of ILC2. Front Immunol. 2013 Oct. 22; 4:334.
Helbig, C., Gentek, R., Backer, R. A., de Souza, Y., Derks, I. A., Eldering, E., Wagner, K., Jankovic, D., Gridley, T., Moerland, P. D., Flavell, R. A., and Amsen, D. (2012) Notch controls the magnitude of T helper cell responses by promoting cellular longevity. Proceedings of the National Academy of Sciences of the United States of America 109, 9041-9046
Kochenderfer J N. et al. Construction and preclinical evaluation of an anti-CD19 chimeric antigen receptor. J Immunother. 2009 September; 32(7): 689-702.
Legrand N. et al. Functional CD47/signal regulatory protein alpha (SIRPα) interaction is required for optimal human T- and natural killer-(NK) cell homeostasis in vivo. J Immunol 2009; 183:7645-7655
Ma, J. S., Kim, J. Y., Kazane, S. A., Choi, S. H., Yun, H. Y., Kim, M. S., Rodgers, D. T., Pugh, H. M., Singer, O., Sun, S. B., Fonslow, B. R., Kochenderfer, J. N., Wright, T. M., Schultz, P. G., Young, T. S., Kim, C. H., and Cao, Y. (2016) Versatile strategy for controlling the specificity and activity of engineered T cells. Proc Natl Acad Sci USA 113, E450-458
Mueller, S. N., Langley, W. A., Carnero, E., Garcia-Sastre, A., and Ahmed, R. (2010) Immunization with live attenuated influenza viruses that express altered NS1 proteins results in potent and protective memory CD8⁺ T-cell responses. J Virol 84, 1847-1855
Morsut, L., Roybal, K. T., Xiong, X., Gordley, R. M., Coyle, S. M., Thomson, M., and Lim, W. A. (2016) Engineering Customized Cell Sensing and Response Behaviors Using Synthetic Notch Receptors. Cell 164, 780-791
Nam, Y., Sliz, P., Pear, W. S., Aster, J. C., and Blacklow, S. C. (2007) Cooperative assembly of higher-order Notch complexes functions as a switch to induce transcription. Proceedings of the National Academy of Sciences of the United States of America 104, 2103-2108.
Nicholson, I. C. et al. Construction and characterisation of a functional CD19 specific single chain Fv fragment for immunotherapy of B lineage leukaemia and lymphoma. Molecular Immunology 1997; 34:1157-1165
Palmer, D. F., Dowdle, W. R., Coleman, M. T., and Schild, G. C. (1975) Advanced laboratory techniques for influenza diagnosis: procedural guide. Haemagglutination inhibition test. in Atlanta: US Department of Health, Education and Welfare, Public Health Service, US Department of Health, Education and Welfare, Public Health Service. pp 25-62
Rodgers, D. T., Mazagova, M., Hampton, E. N., Cao, Y., Ramadoss, N. S., Hardy, I. R., Schulman, A., Du, J., Wang, F., Singer, O., Ma, J., Nunez, V., Shen, J., Woods, A. K., Wright, T. M., Schultz, P. G., Kim, C. H., and Young, T. S. (2016) Switch-mediated activation and retargeting of CAR-T cells for B-cell malignancies. Proc Natl Acad Sci USA 113, E459-468
Roybal, K. T., Rupp, L. J., Morsut, L., Walker, W. J., McNally, K. A., Park, J. S., and Lim, W. A. (2016) Precision Tumor Recognition by T Cells With Combinatorial Antigen-Sensing Circuits. Cell 164, 770-779
Topham, D. J., Castrucci, M. R., Wingo, F. S., Belz, G. T., and Doherty, P. C. (2001) The role of antigen in the localization of naive, acutely activated, and memory CD8(+) T cells to the lung during influenza pneumonia. J Immunol 167, 6983-6990
Van der Sluijs, K. F., van Elden, L. J., Nijhuis, M., Schuurman, R., Pater, J. M., Florquin, S., Goldman, M., Jansen, H. M., Lutter, R., and van der Poll, T. (2004) IL-10 is an important mediator of the enhanced susceptibility to pneumococcal pneumonia after influenza infection. J Immunol 172, 7603-7609
van Lent A U, et al. IL-7 enhances thymic human T cell development in “human immune system” Rag2−/−IL-2Rgammac−/− mice without affecting peripheral T cell homeostasis. J Immunol. 2009; 183:7645-7655
Wherry, E. J., and Kurachi, M. (2015) Molecular and cellular insights into T cell exhaustion. Nat Rev Immunol 15, 486-499
Young, M. D., Wakefield, M. J., Smyth, G. K., and Oshlack, A. (2010) Gene ontology analysis for RNA-seq: accounting for selection bias. Genome biology 11, R14.

1. A chimeric receptor comprising an intracellular domain, a transmembrane domain, a heterodimerization domain and a Lin-12-Notch (LNR) repeats domain of a Notch receptor, and a heterologous extracellular ligand-binding domain.

2. The chimeric receptor according to claim 1 wherein the receptor is capable of Notch signaling.

3. The chimeric receptor according to claim 1 wherein said heterologous extracellular ligand-binding domain is selected from the group consisting of:
a ligand binding domain specific for a soluble ligand;
a ligand binding domain specific for a cell surface antigen, such as a ScFv antibody domain, preferably a ScFv antibody domain that is specific for a tumor cell surface antigen;
an extracellular ligand-binding domain of an Fc receptor or a ligand-binding fragment thereof;
an extracellular domain that comprises an epitope for an antibody that can crosslink the chimeric receptor without involvement of a surface molecule.
an extracellular domain that comprises a moiety, such as biotin, that can be crosslinked by an agent with multiple binding sites for that moiety, such as streptavidin.

4. The chimeric receptor according to claim 1 further comprising a linking sequence located between the LNR domain and the heterologous extracellular ligand-binding domain.

5. A nucleic acid molecule comprising a sequence encoding a chimeric receptor according to claim 1.

6. (canceled)

7. An isolated cell comprising the nucleic acid molecule according to claim 5.

8. The cell according to claim 7, wherein said cell is an immune cell, such as a natural killer cell, macrophage, neutrophil, eosinophil, or T cell, such as a tumor derived T cell or a tumor infiltrating lymphocyte (TIL).

9. The cell according to claim 7 wherein said cell is an autologous T cell isolated from a patient suffering from cancer.

10. The cell according to claim 7 wherein said cell expresses a chimeric receptor according to claim 1, preferably wherein said cell further expresses a chimeric antigen receptor.

11. (canceled)

12. A pharmaceutical composition comprising the nucleic acid molecule according to claim 5, and a pharmaceutically acceptable carrier, diluent or excipient.

13. A method for improving T cell function and/or T cell survival in a subject in need thereof, the method comprising administering to the subject a therapeutically effective amount of a chimeric receptor according to claim 1.

14. (canceled)

15. The method according to claim 13, wherein said method comprises preventing or inhibiting T cell exhaustion.

16. The method according to claim 13, wherein said method comprises immunotherapy of said subject.

17. (canceled)

18. The method according to claim 16 wherein said immunotherapy further comprises antibody-based immunotherapy.

19. The method according to claim 13 wherein said subject is suffering from cancer and the method comprises treating cancer in said subject.

20. The method according to claim 13, comprising enhancing efficacy of an antibody-based immunotherapy in a subject suffering from cancer and being treated with said antibody.

21-23. (canceled)

24. The method according to claim 19 wherein said method comprises:
isolating T cells from the subject;
modifying said T cells by providing them with a nucleic acid sequence encoding the chimeric receptor according to claim 1;
returning the modified T cells to the subject.

25. A method of producing a population of cells according to claim 7, comprising
providing cells, preferably human T-cells,
providing said cells with a nucleic acid molecule according to claim 5, and
allowing expression of the chimeric antigen receptor according to claim 1.

26. The cell according to claim 7, which is a genetically modified T cell that is transduced by the nucleic acid molecule according to claim 5.

27. The cell according to claim 26, which is transduced by a vector comprising the nucleic acid molecule.

28. The method according to claim 13, wherein said chimeric receptor is administered to the subject by administering a therapeutically effective amount of the nucleic acid molecule according to claim 5.

29. The method according to claim 13, wherein said chimeric receptor is administered to the subject by administering a therapeutically effective amount of a vector comprising the nucleic acid molecule according to claim 5.

30. The method according to claim 13, wherein said chimeric receptor is administered to the subject by administering a therapeutically effective amount of cells according to claim 7.


SEQUENCE LISTING (12 sequences)

SEQ ID NO: 1 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 9 aa, Influenza A virus
ASNENMETM

SEQ ID NO: 3 - DNA, 22 bp, Artificial Sequence
  primer
caaagcgtctacgctgcagtcc

SEQ ID NO: 4 - DNA, 23 bp, Artificial Sequence
  primer
tttgtgttcacgctcaccgtgcc

SEQ ID NO: 5 - DNA, 23 bp, Artificial Sequence
  probe
aagaccaatcctgtcacctctga

SEQ ID NO: 6 - DNA, 39 bp, Artificial Sequence
  primer
gatcaggaattccacaccatgggagatccacgaaatgaa

SEQ ID NO: 7 - DNA, 32 bp, Artificial Sequence
  primer
gatcaggatatccaccttcctcttcttcttgg

SEQ ID NO: 8 - DNA, 33 bp, Artificial Sequence
  primer
atcggaggttctcgcaagcgccggcggcagcat

SEQ ID NO: 9 - DNA, 40 bp, Artificial Sequence
  primer
gatcagaagcttgaattcttacttgaaggcctccggaatg

SEQ ID NO: 10 - protein, 20 aa, Homo sapiens
MLLLVTSLLLCELPHPAFLL

SEQ ID NO: 11 - protein, 250 aa, Artificial Sequence
  Ig heavy chain Variable domain of FMC63-28Z anti CD19 ScFv
IPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAA

SEQ ID NO: 12 - protein, 8 aa, Gallus gallus
SIINFEKL